(19) United States
(12) Patent Application Publication
(10) Pub. No.: US 2024/0003894 A1
(43) Pub. Date: Jan. 4, 2024

(54) TRYPTOPHAN-CONTAINING CHEMIGENETIC FLUORESCENT INDICATOR

(71) Applicant: HOWARD HUGHES MEDICAL INSTITUTE, Chevy Chase, MD (US)

(72) Inventors: Eric R. Schreiter, Leesburg, VA (US); Helen Farrants, Ashburn, VA (US)

(21) Appl. No.: 18/318,997

(22) Filed: May 17, 2023

Related U.S. Application Data
(60) Provisional application No. 63/342,892, filed on May 17, 2022.

(51) Int. Cl. G01N 33/58; C07K 14/47

(52) U.S. Cl. CPC G01N 33/582; G01N 2458/00; C07K 14/4728

(57) ABSTRACT

Tryptophan-containing chemigenetic fluorescent indicators for detecting biologically-relevant analytes are described and are useful for detecting analytes in a living animal.

### RELATED APPLICATIONS

This application claims priority from U.S. Provisional Application Ser. No. 63/342,892 filed May 17, 2022, the entire disclosure of which is incorporated herein by this reference.

### TECHNICAL FIELD

The presently-disclosed subject matter generally relates to indicators for biologically-relevant analytes, such as calcium or glucose. In particular, certain embodiments of the presently-disclosed subject matter relate to tryptophan-containing chemigenetic fluorescent indicators for use in detecting biologically-relevant analytes in a living animal.

### INTRODUCTION

Chemigenetic fluorescent indicators have become useful reagents in imaging dynamic cellular activity. Chemigenetic fluorescent indicators, also known as chemigenetic, hybrid or semisynthetic indicators, combine a fluorescent dye, a self-labeling protein, and analyte sensing domains (1, 2). Conformational changes in the analyte sensing domain occur when the analyte-of-interest binds to the analyte sensing domain, which alter the fluorescent properties of the fluorescent dye.

In chemigenetic fluorescent indicators that make use of a small molecule fluorescent dye, as opposed to a fluorescent protein, the conformational changes to the analyte sensing domain are similar to processes that have been reported to occur in genetically encoded fluorescent indicators (GEFI) based on fluorescent proteins (3). The small-molecule fluorescent dye can offer certain advantages over fluorescent protein-based indicators, such as access to a wider range of spectral properties, and a larger quantum yield (4, 5). However, only a limited number of chemigenetic fluorescent indicators have been employed in living animals compared to the fluorescent protein-based indicators, as delivery of the small-molecule dye has been challenging (6).

Accordingly, there remains a need in the art for chemigenetic fluorescent indicators that can be used to detect a biologically-relevant analytes of interest in a living animal.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes chemigenetic fluorescent indicators for detecting an analyte. Embodiments of the chemigenetic fluorescent indicator can be used to detect an analyte in a living animal. The presently-disclosed subject matter further includes methods of using the chemigenetic fluorescent indicator to detect an analyte, such as a method of detecting an analyte in a living animal.

Indicators as disclosed herein include a self-labeling protein (SLP) having a tryptophan (W)-modification, which is provided together with an analyte sensing domain (ASD) in a fusion protein. The indicators further include a bioavailable fluorescent dye conjugated to a ligand for the SLP. The indicator is assembled when the ligand is bound to the SLP, thereby attaching the fluorescent dye to the indicator. When an analyte-of-interest binds to the ASD, a conformational change occurs. The proximity of the W-modification to the fluorescent dye in the assembled indicator is such that the conformational change causes the W-modification either to begin or cease quenching fluorescence.

In some embodiments, the indicator can be positive going, such that the conformational change upon analyte-binding to the ASD causes the W-modification to cease quenching fluorescence. An exemplary positive-going indicator is depicted in FIG. 1A. On the left, prior to assembly, the fluorescence is emitted from the dye, as indicated by the dark grey six-membered rings. In the middle, once the indicator is assembled, the W-modification is positioned such that fluorescence is quenched, as indicated by the white six-membered rings. On the right, upon analyte-binding to the ASD, there is a conformational change in the ASD such that the W-modification is no longer quenching fluorescence, as indicated by the dark grey six-membered rings. Accordingly, when the indicator is positive going, increasing fluorescence indicates increasing amounts of the analyte of interest.

In some embodiments, the indicator can be negative going, such that the conformational change upon analyte-binding to the ASD causes the W-modification to quench fluorescence. An exemplary negative-going indicator is depicted in FIG. 1B. On the left, prior to assembly, the fluorescence is emitted from the dye, as indicated by the dark grey six-membered rings. In the middle, once the indicator is assembled, the W-modification is positioned such that fluorescence continues to be emitted from the dye. On the right, upon analyte-binding to the ASD, there is a conformational change in the ASD such that the W-modification quenches fluorescence, as indicated by the white six-membered rings. Accordingly, when the indicator is negative going, decreasing fluorescence indicates increasing amounts of the analyte of interest.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A and 1B. Schematic Representation of Tryptophan-Containing Chemigenetic Indicators. The components highlighted in the figures include: an analyte sensing domain (ASD) (“sensor domain” in the schematic), a mutant of a self-labeling protein (SLP) (e.g., HaloTag®) containing tryptophan (W), which reacts with an SLP ligand that is conjugated to a small-molecule fluorescent dye to assemble the indicator. When an analyte-of-interest binds to the ASD, conformation changes occur in that ASD, which modulates the fluorescent emission of the small-molecule fluorescent dye. The sensor can be positive going such that binding of the analyte results in an increase in fluorescence (FIG. 1A), or negative going such that binding of the analyte results in a decrease in fluorescence (FIG. 1B).

FIG. 2A. Depiction of exemplary portion of a chemigenetic indicator, including a self-labeling protein (SLP), depicted in grey, and a fluorescent dye conjugated to a ligand that is within the ligand binding site of the SLP. A portion of the SLP that is appropriately close to the fluorescent dye is identified and depicted in light gray.

FIG. 2B. Examples of small-molecule fluorescent dyes conjugated to self-labeling protein (SLP) ligands (pictured example includes a HaloTag® ligand), which can be used in accordance with the presently-disclosed subject matter.

FIG. 3A-3C. Series of titration curves for of analysts-of-interest (calcium ions or glucose) with different examples of tryptophan-containing chemigenetic fluorescent indicators made in accordance with the presently-disclosed subject matter. FIG. 3A. Insertion topologies of calcium sensors with different insertion sites of calcium sensing domains, different calmodulin binding peptide, an example of a negative going sensor, and an example of tuning the affinity for calcium using mutations on the calmodulin-binding peptide. FIG. 3B. Circular permuted HaloTag topologies for building calcium indicators. FIG. 3C. Glucose indicators based on insertion topologies.

FIG. 4. Fluorescent lifetime imaging in cultured rat hippocampal neurons expressing WHaloCaMP1a and labeled with JF669-HTL. Scale bar 50 μm.

FIGS. 5A and 5B. FIG. 5A includes images from fluorescence imaging of cultured rat hippocampal neurons expressing WHaloCaMP1a, 1b or 1c, labeled with JF669-HTL. Scale bar 50 μm. FIG. 5B includes fluorescent emission from cultured rat hippocampal neurons expressing WHaloCaMP1a, 1b or 1c from eliciting action potentials from a field stimulation assay. Number of action potentials are: 1, 2, 3, 5, 10, 20, 40, 80, 120, 160. Black line is the mean of 30-80 neurons, with standard error of the mean in grey.

FIG. 6A-6D. Fluorescence imaging of cultured rat hippocampal neurons expressing WHaloCaMP1a labeled with different small-molecule fluorescent dye-ligands, and fluorescent emission from elicited action potentials (APs) in an electric field stimulation assay with 1, 2, 3, 5, 10, 20, 40, 80, 120, 160 APs. Black line is the mean of 30-80 neurons, with standard error of the mean in grey. Scale bar 50 μm. Small-molecule dye ligand: FIG. 6A. JF525-HTL, FIG. 6B: JF552-HTL, FIG. 6C: JF608-HTL, FIG. 6D: JF722-HTL.

FIG. 7A-7C. Fluorescence imaging of cultured rat hippocampal neurons expressing WHaloCaMP1b labeled with different small-molecule fluorescent dye-ligands, and fluorescent emission from elicited action potentials (APs) in an electric field stimulation assay with 1, 2, 3, 5, 10, 20, 40, 80, 120, 160 APs. Black line is the mean of 30-80 neurons, with standard error of the mean in grey. Scale bar 50 μm. Small-molecule dye ligand: FIG. 7A. JF525-HTL, FIG. 7B: JF552-HTL, FIG. 7C: JF608-HTL.

FIGS. 8A and 8B. In vivo calcium imaging in zebrafish larvae (5 days post fertilization). FIG. 8A. fluorescent micrograph of WHaloCaMP-JF552 imaged on a light sheet microscope with 561 nm excitation, segmented cells using cellpose activity traces calculated with suite2p and example traces of five neurons. FIG. 8B. fluorescent micrograph of WHaloCaMP-JF669 imaged on a light sheet microscope with 638 nm excitation, segmented cells using cellpose, activity traces calculated with suite2p and example traces of five neurons. Scale bar 100 μm.

FIG. 9A-9C. Simultaneous in vivo calcium imaging of astrocyte and neuronal activity in zebrafish larvae (4 days post fertilization). FIG. 9A. Max projections of evalv3: WHaloCaMP-JF669-GFP, gfap:jRGECO1b fish imaged on a light sheet with 638 nm, 561 nm and 488 nm excitation. Scale bar 100 μm. FIG. 9B. elavl3:WHaloCaMP-JF669 fluorescent image and segmented cells using cellpose, with fluorescent traces from suite2p from over 1000 neurons in the hind brain of the zebrafish larvae. FIG. 9C. gfap:jRGECO1b fluorescent image and segmented cells using cellpose, as well as activity traces from the same time interval as in FIG. 9B. FIG. 9B and FIG. 9C, scale bar 50 μm.

FIGS. 10A and 10B. In vivo calcium imaging in a mouse expressing WHaloCaMP1b-GFP in the visual cortex, labeled with JF669-HTL after retro orbital injection. FIG. 10A. Imaging with a 2-photon microscope with adaptive optics using 1225 nm excitation of WHaloCaMP1b-JF669-HTL and 950 nm excitation for GFP. Max projection of around 400 μm. FIG. 10B. activity imaging of neurons in the visual cortex of mouse segmented using cellpose and suite2p.

### REFERENCE TO AN ELECTRONIC SEQUENCE LISTING

The contents of the electronic sequence listing (Schreiter 22012US.xml; Size: 27.2 KB; and Date of Creation: May 15, 2023) is herein incorporated by reference in its entirety.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes chemigenetic fluorescent indicators for detecting an analyte. Embodiments of the chemigenetic fluorescent indicator can be used to detect an analyte in a living animal. The presently-disclosed subject matter further includes methods of using the chemigenetic fluorescent indicator to detect an analyte, such as a method of detecting an analyte in a living animal.

In some embodiments, the chemigenetic fluorescent indicator includes an analyte sensing domain (ASD), a self-labeling protein (SLP) having a tryptophan (W)-modification, provided together with the ASD in a fusion protein, and a fluorescent dye conjugated to a ligand for the SLP. Upon assembly of the indicator, and binding of an analyte, conformational changes in the analyte sensing domain alters the fluorescent properties of the fluorescent dye, such that detection of the analyte can be made based on a change in fluorescence.

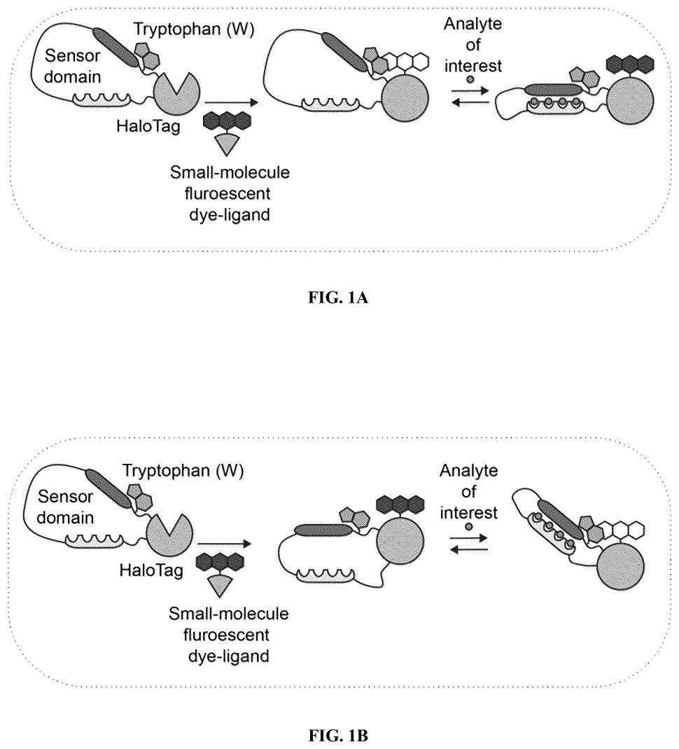

In some embodiments, the sensor can be positive going such that binding of the analyte results in an increase in fluorescence. With reference to FIG. 1A, in positive going embodiments, the W-modification quenches fluorescence when the analyte-of-interest is not bound to the ASD. However, when the analyte-of-interest binds to the ASD, fluorescence is observed.

In some embodiments, the sensor can be negative going such that binding of the analyte results in a decrease in fluorescence. With reference to FIG. 1B, in negative going embodiments, fluorescence is observed when the analyte-of-interest is not bound to the ASD. However, when the analyte-of-interest binds to the ASD, the W-modification quenches fluorescence.

The self-labeling protein (SLP) as used herein can be selected from among those known in the art, with modifications as disclosed herein. Examples of self-labeling proteins that can be used in accordance with the presently-disclosed subject matter include, but are not limited to, HaloTag®, SNAP-Tag®, TMP-Tag®, βLac-tag, CLIP-Tag®, TMP-Tag®, and biotin-avidin.

Referring again to FIGS. 1A and 1B, among the modifications disclosed herein is the W-modification, which can quench the fluorescence of the fluorescent dye, depending on the conformational changes in the ASD that occur in the presence of the analyte-of-interest.

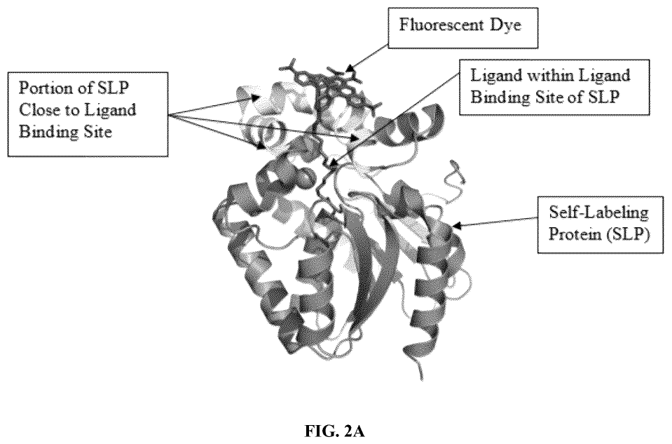

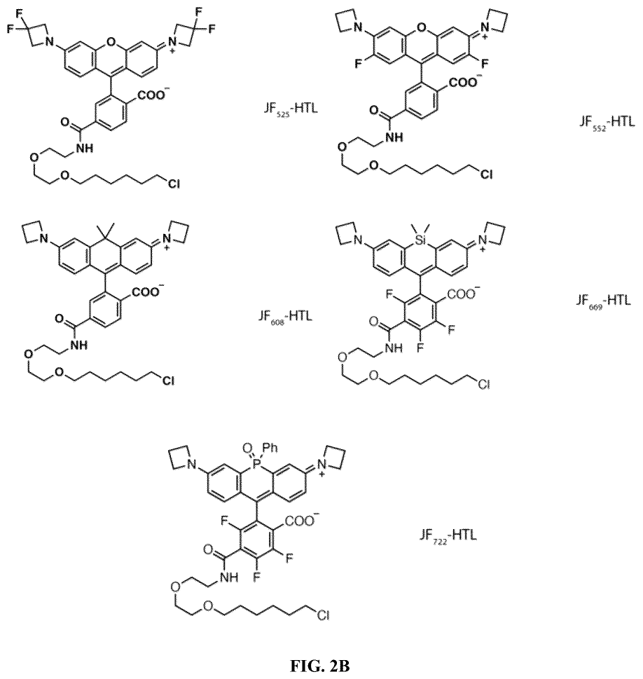

While such conformational changes allow the W-modification to quench (or not quench) the fluorescence, as will be appreciated by the skilled artisan, to achieve such quenching, the W-modification to the SLP is placed close to the ASD, and close to the fluorescent dye in the assembled indicator. In this regard, reference is made to FIG. 2A, in which a portion of the SLP that is appropriately close to the fluorescent dye is identified and depicted in light gray. In some embodiments, the W-modification in the SLP can be within about 1, 2, 3, 4, 5, 6, 7, or 8 Å from the fluorescent dye.

In some embodiments, the SLP has the sequence of SEQ ID NO: 1, and the W-modification can be within residues 145 to 180. In some embodiments, the tryptophan-modification is between residues 145 and 176. In some embodiments, the tryptophan-modification is at residue 151. In some embodiments, the tryptophan-modification is at residue 171.

In some embodiments, the SLP has the sequence of SEQ ID NO: 1; provided as an N-terminal portion and a C-terminal portion; wherein 0, 1, 2, 3, 4, or 5 residues are removed from a terminus of the N-terminal portion, the C-terminal portion, or both; and wherein there are 1, 2, 3, 4, or 5 mutations between residues 145 and 180.

With continued reference to SEQ ID NO: 1, in some embodiments of the indicator, there is at least one additional mutation at a residue selected from the group consisting of: 157, 158, 176, 178, and 180. In some embodiments, the at least one additional mutation is selected from the group consisting of: V157L, G158D, G176A, V178A, V178I, G178W, P180Y, P180T, and P180V.

In some embodiments of the indicator, the ASD and/or a linker is disposed between the N-terminal and C-terminal portions of the SLP.

Linkers of various lengths can be used in embodiments of the indicator, and include linkers of 2 amino acids to about 20 amino acids. Embodiments of linkers include, but are not limited to, GGS linkers that are twelve amino acids (SEQ ID NO: 16), fifteen amino acids (SEQ ID NO: 17, or nineteen amino acids (SEQ ID NO: 18). In some embodiments, shorter linkers of two amino acids (EV) or three amino acids (GGS, LLS) can be used. In some embodiments, multiple linkers are used, as described further herein.

In some embodiments of the indicator, the N-terminal portion of the SLP extends from residue 1-4 to residue 150-180; and the C-terminal portion of the SLP extends from residue 151-181 to 294-297.

In some embodiments of the indicator, the SLP is circularly permutated. Circular permutation of proteins, such as SLPs, is known in the art and refers to a rearrangement of protein domains. Exemplary embodiments are described in the Examples (cp WHaloCaMP1a and cpWHaloCaMP1b). In some embodiments, there is a linker is disposed between the rearranged C-terminal and the N-terminal portions of the circularly permutated SLP.

Embodiments of the indicator can be provided for use in detecting any analyte-of-interest. Often, the analyte will be one that is biologically-relevant for detection in a living animal. Examples of such analytes include, but are not limited to calcium (Ca²⁺), glucose, GABA, DA, NE, opioids, 5-HT, MT, Ach, maltose, Tre, ATP, GTP, cAMP, cGMP, ADP, citrate, pyruvate, NAD⁺/NADH, NADPH, PKA, PKC, Pn, nicotine, H₂O₂, NH⁴⁺, and Zn²⁺.

Depending on the analyte-of-interest, an appropriate ASD will be selected, which undergoes a conformational change when it binds to an analyte. Examples of such ASD are known in the art, for example, reference is made to Nasu et al. (13), which is incorporated herein by this reference.

In some embodiments of the indicator, the analyte is Ca²⁺. In some embodiments of the indicator, the ASD comprises calmodulin and a calmodulin binding peptide. In some embodiments there is a linker disposed between the calmodulin and a calmodulin binding peptide.

The calcium-binding ASDs can include any suitable domain or domains for binding calcium and influencing the fluorescence of the fluorescent dye. For example, in other embodiments, the calcium-binding domain(s) can include, but are not limited to, troponin C, calbindin, calretinin, centrin, any other suitable calcium-binding protein, and/or a combination thereof, along with the associated binding peptide(s) (e.g., calretinin binding peptide for calretinin).

In some embodiments of the indicator, the analyte glucose. In some embodiments of the indicator, the ASD comprises glucose-binding MgIB. In some embodiments, there are linkers flanking the ASD.

The fluorescent dye as used herein can be selected from among those known in the art, including fluorescent proteins and small molecules (fluorescent dyes that are not proteins).

In some embodiments, the fluorescent dye is selected because it is capable of delivery over biological membranes, in which case it is a bioavailable fluorescent dye. How small molecules are delivered across biological membranes in living animals is still somewhat of an empirical science, and a dye must be tested before use; however, Grimm et al. (2020) [7] outlines a rubric for dyes that show “improved permeability” depending on a measured equilibrium between a rhodamine zwitterion and lactone state.

In some embodiments, the selected fluorescent dye has a measured log K_{L-Z} of 0 to −2, and had been shown to have promising in vivo properties, e.g., reaching the brain of mice after retroorbital injection of the dye.

FIG. 2B includes examples of small-molecule fluorescent dyes conjugated to self-labeling protein (SLP) ligands (pictured example includes a HaloTag® ligand), which can be used in accordance with the presently-disclosed subject matter.

As noted herein, the presently-disclosed subject matter further includes a method of detecting an analyte-of-interest in a cell, which involves contacting the cell with the indicator of as disclosed herein.

As also noted here, the presently-disclosed subject matter is useful for detecting an analyte in a living animal. In this regard, the cell can be in the living animal, and the indicator can be administered to the living animal. The methods can further involve detecting changes in fluorescence, thereby detecting the analyte in the cell and/or animal.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

Several small-molecule dyes have been described with properties that favor delivery over biological membranes in living animals (7). Described in these Examples is the production of embodiments of chemigenetic fluorescent indicators for use in detecting biologically-relevant analytes in a living animal. As described herein, upon binding the biologically relevant analytes, there is a modulation in the fluorescence of the small-molecule dyes when they are covalently anchored to a self-labeling protein, e.g., HaloTag (8), which is fused to analyte sensing domain. It has also recently been shown that tryptophan can modulate the fluorescent properties of dyes when bound to HaloTag (9). The unique indicators as disclosed herein make use of tryptophan modifications in a particular manner to benefit the efficacy of the disclosed chemigenetic fluorescent indicators.

By adding tryptophan to specific positions in the HaloTag protein, and placing sensing domains close to the tryptophan residue and small-molecule fluorescent dye ligand (FIG. 1A-1B), embodiments of chemigenetic fluorescent indicators were built. Tables IA-1C identify chemigenetic fluorescent indicators, which were built and tested in these Examples.

TABLE 1A

| | Self-Labeling Protein (SLP) | |
|---|---|---|
| Examples of | ASD disposed between N-Terminal and C- | Analyte sensing |
| Indicators | Terminal Portions of SLP | domain (ASD) |
| WHaloCaMP1a | SEQ ID NO: 1 with the following mutations: | SEQ ID NO: 2 |
| | Tryptophan-modification: | SEQ ID NO: 3 |
| | G171W | GGS linker disposed |
| | Mutations found to improve response of sensor: | between SEQ ID NO: 2 |
| | G176A | and 3. |
| | V178A | |
| | P180Y | |
| | N-Terminal - 1-179 | |
| | C-Terminal - 180-297 | |
| WHaloCaMP1b | SEQ ID NO: 1 with the following mutations: | SEQ ID NO: 2 |
| | Tryptophan-modification: | SEQ ID NO: 3 |
| | A151W | GGS linker disposed |
| | Mutation found to improve response of sensor: | between SEQ ID NO: 2 |
| | G158D | and 3. |
| | N-Terminal - 1-154 | |
| | C-Terminal - 157-297 | |
| WHaloCaMP1c | SEQ ID NO: 1 with the following mutations: | SEQ ID NO: 2 |
| | Tryptophan-modification: | SEQ ID NO: 4 |
| | G171W | GGS linker disposed |
| | Mutation found to improve response of sensor: | between SEQ ID NO: 2 |
| | G176A | and 4. |
| | V178I | |
| | P180T | |
| | N-Terminal - 1-179 | |
| | C-Terminal - 180-297 | |
| WHaloCaMP1d | SEQ ID NO: 1 with the following mutations: | SEQ ID NO: 2 |
| | Tryptophan-modification: | SEQ ID NO: 3 |
| | A151W | GGS linker disposed |
| | N-Terminal - 1-153 | between SEQ ID NO: 2 |
| | C-Terminal - 158-297 | and 3. |
| WHaloCaMP1e | Tryptophan-modification: | SEQ ID NO: 2 |
| (SEQ ID NO: 6) | G171W | SEQ ID NO: 3 with the |
| | Mutation found to improve response of sensor: | following mutation: |
| | G176A | V12T |
| | V178A | GGS linker disposed |
| | P180Y | between SEQ ID NO: 2 |
| | N-Terminal - 1-179 | and 3. |
| | C-Terminal - 180-297 | |

TABLE 1B

| Examples of | Self-Labeling Protein (SLP) | |
|---|---|---|
| Indicators | Linker disposed between | |
| Circular | N-Terminal and C- | Analyte sensing |
| permutation | Terminal Portions of SLP | domain (ASD) |
| cpWHaloCaMP1a | Tryptophan-modification: | SEQ ID NO: 2 |
| (SEQ ID NO: 8) | G171W | SEQ ID NO: 3 |
| | Mutation found to improve | SLP and linker |
| | response of sensor: | disposed between |
| | P180V | SEQ ID NO: 2 and 3. |
| | G178W | |
| | C-Terminal - 180-296 | |
| | N-Terminal - 2-179 | |
| cpWHaloCaMP1b | Tryptophan-modification: | SEQ ID NO: 2 |
| (SEQ ID NO: 10) | A151W | SEQ ID NO: 3 |
| | Mutation found to improve | SLP and linker |
| | response of sensor: | disposed between |
| | V157L | SEQ ID NO: 2 and 3. |
| | G158D | |
| | C-Terminal - 157-296 | |
| | N-Terminal - 2-154 | |

TABLE 1C

| Examples of | Self-Labeling Protein (SLP) | |
|---|---|---|
| Indicators | ASD disposed between N-Terminal | Analyte sensing |
| Gluco | and C-Terminal Portions of SLP | domain (ASD) |
| WHaloGluco1a | Tryptophan-modification: | SEQ ID NO: 5 |
| (SEQ ID NO: 12) | A151W | Flanked by |
| | N-Terminal - 1-154 | linkers. |
| | C-Terminal - 157-297 | |
| WHaloGluco1b | Tryptophan-modification: | SEQ ID NO: 5 |
| (SEQ ID NO: 14) | G171W | Flanked by |
| | N-Terminal - 1-179 | linkers. |
| | C-Terminal - 180-297 | |

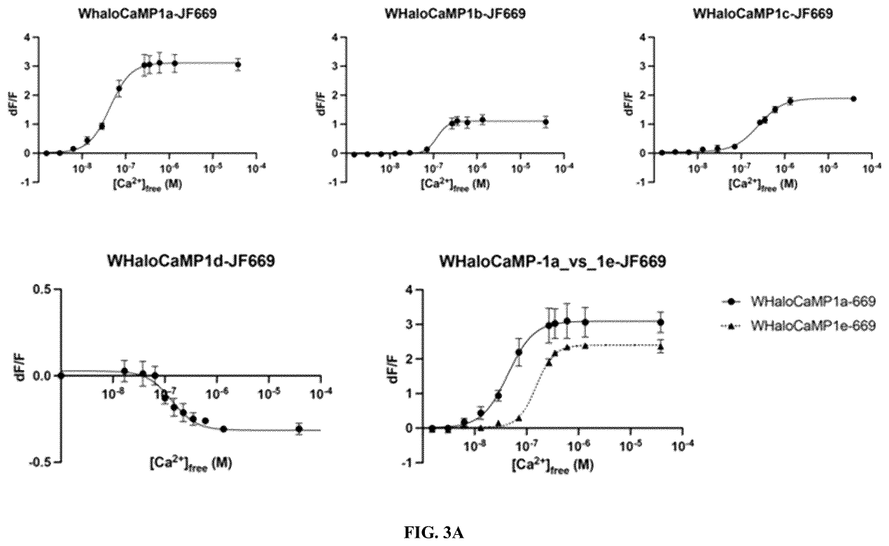

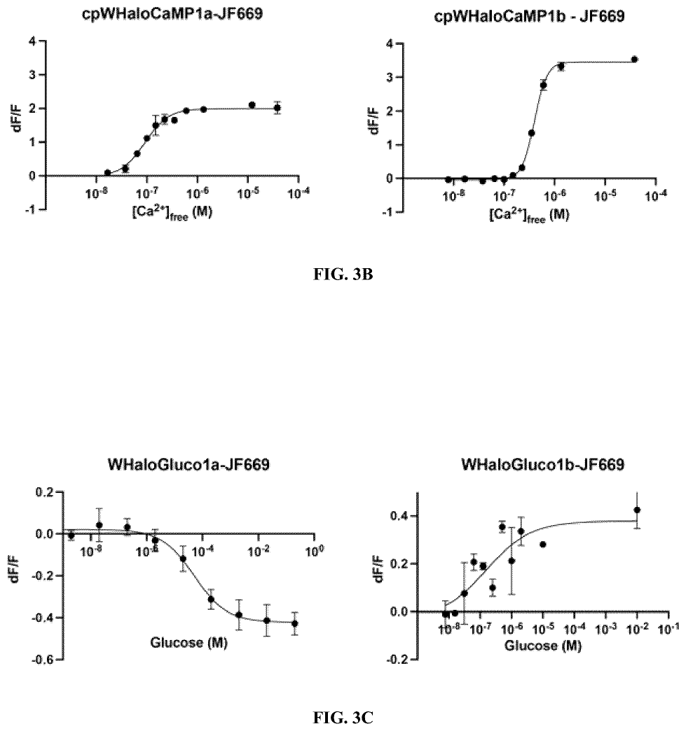

The chemigenetic fluorescent indicators were tested for calcium ions and glucose (FIG. 3A-3C). For the calcium indicators the properties could be altered using different insertion sites for the calcium sensor domain, different calmodulin binding peptide (as seen in jGCaMP8 (10)), and rational point mutations on the calmodulin binding peptide (as seen with CAMPARI2 (11)). Variants of the calcium indicators where HaloTag is circularly permuted were also built and tested.

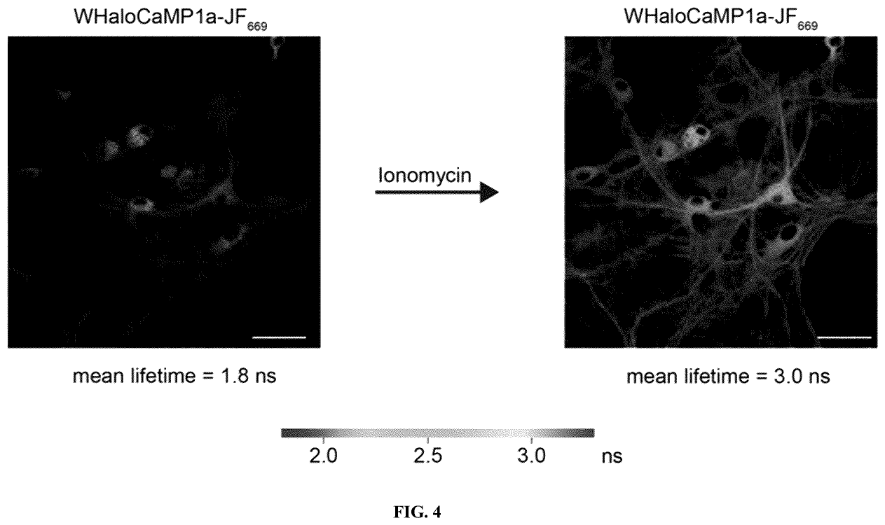

The exemplary chemigenetic fluorescent indicators were characterized in purified protein, in cultured hippocampal neurons, in living zebrafish larvae and in mouse cortex. The chemigenetic fluorescent indicators for calcium ions were shown to work with a change in quantum yield of the small-molecule dye (Table 2), which also leads to a change in fluorescent lifetime (FIG. 4, Table 3).

TABLE 2
in vitro properties of WHaloCaMP1a-JF669 and WHaloCaMP1b-JF669.

| | | | Dynamic | | | |
|---|---|---|---|---|---|---|
| | Ex/Emₐₚₒ | Ex/Emₛₐₜ | range | K_{d} | Hill | k_{off} |
| Sensor | (nm) | (nm) | (Fₘₐₓ/Fₘᵢₙ) | (nM) | coeff | (s⁻¹) |
| WHaloCaMP1a-JF₆₆₉ | 675/687 | 678/689 | 4.1 ± 0.2 | 38 ± 2 | 2.0 | 0.15 ± 0.01 |
| | | | | | | (slow component, 57%) |
| | | | | | | 2.3 ± 0.01 |
| | | | | | | (fast component 43%) |
| WHaloCaMP1b-JF₆₆₉ | 676/686 | 676/686 | 2.1 ± 0.2 | 121 ± 20 | 3.3 | 0.41 ± 0.01 |

| | εₐₚₒ (×1000) | εₛₐₜ (×1000) | | | Brightnessₐₚₒ | Brightnessₛₐₜ |
|---|---|---|---|---|---|---|
| Sensor | (M⁻¹ cm⁻¹) | (M⁻¹ cm⁻¹) | Φₐₚₒ | Φₛₐₜ | (mM⁻¹ cm⁻¹) | (mM⁻¹ cm⁻¹) |
| WHaloCaMP1a-JF₆₆₉ | 131 | 144 | 0.18 | 0.42 | 23.6 | 60.5 |
| WHaloCaMP1b-JF₆₆₉ | 103 | 101 | 0.14 | 0.25 | 14.4 | 25.2 |

TABLE 3
Fluorescent lifetime properties of WHaloCaMP1a-
JF669 in purified protein.

| | | Lifetimes | | Mean lifetime |
|---|---|---|---|---|
| | Components | (ns) | weight | (ns) |
| EGTA | 2 | 0.7 | 38% | 1.9 |
| (no Ca²⁺) | | 2.6 | 62% | |
| Ca²⁺ | 2 | 0.9 | 12% | 3.1 |
| | | 3.4 | 88% | |

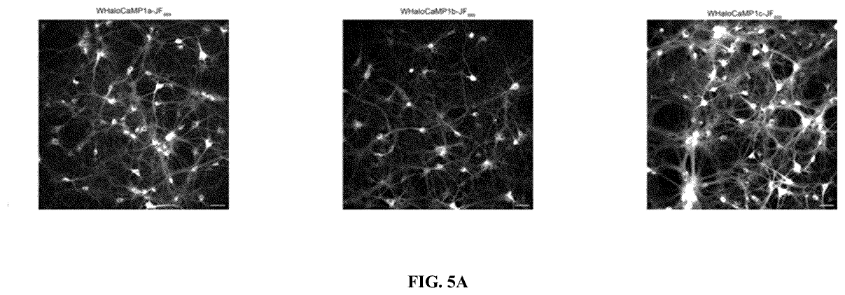

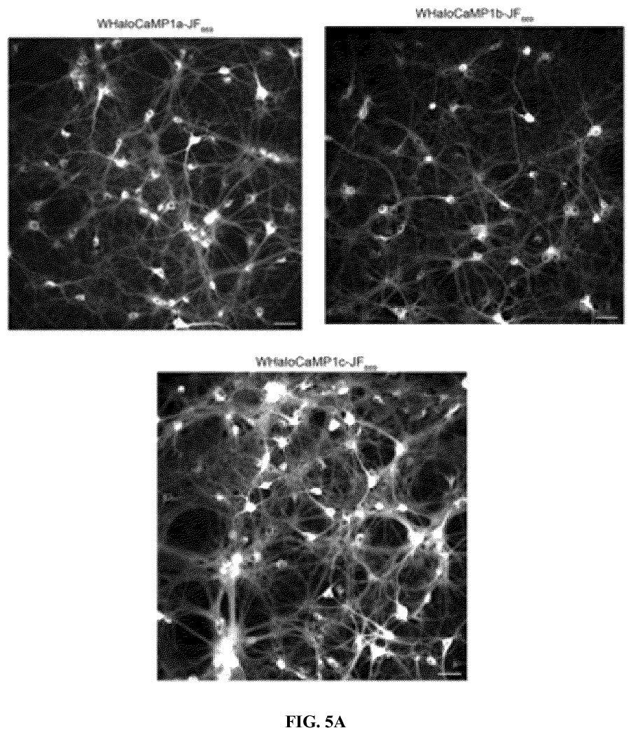

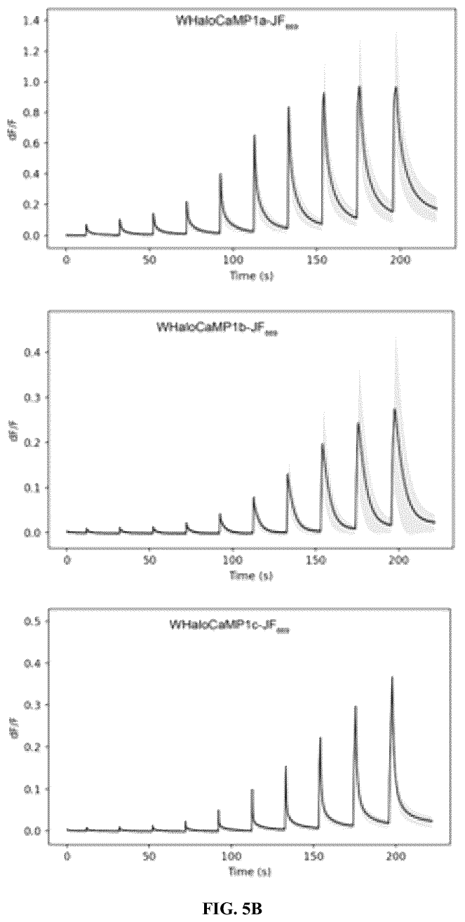

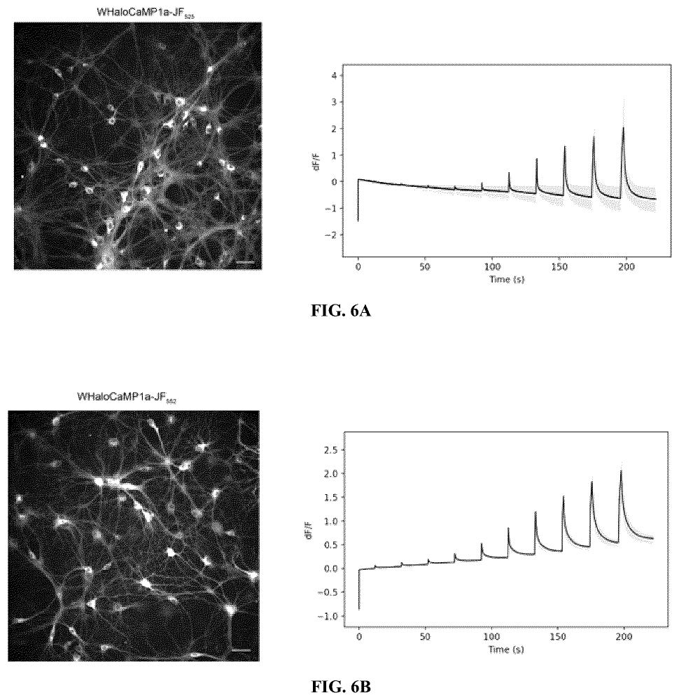

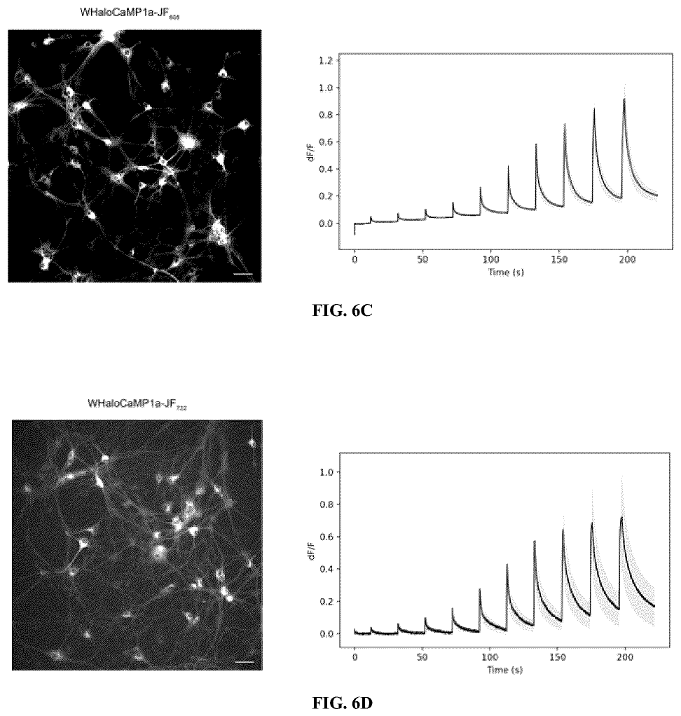

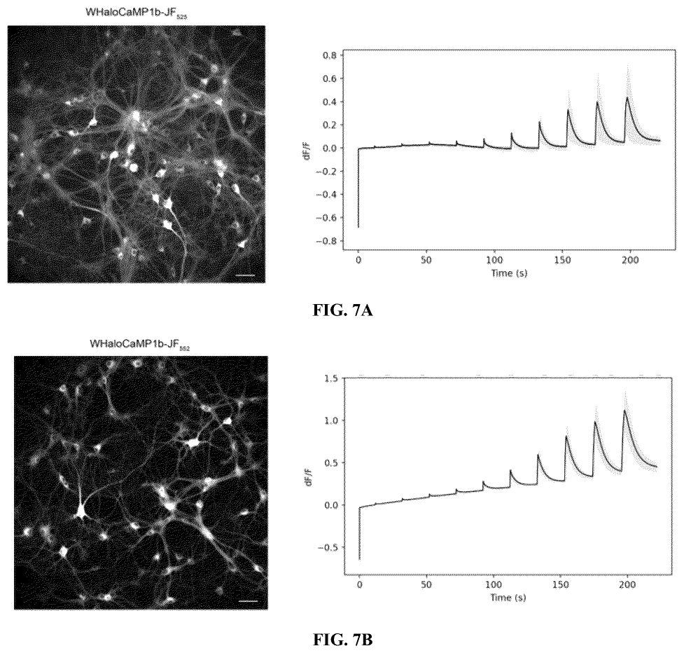

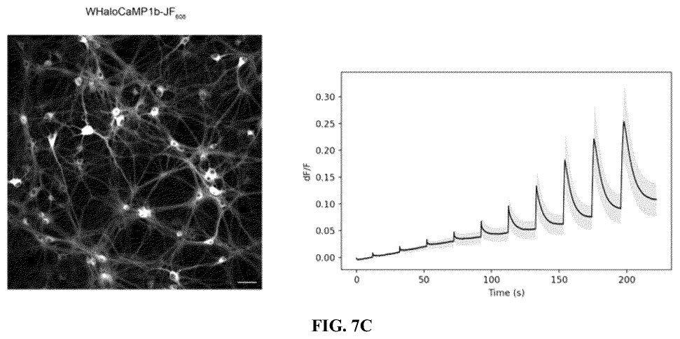

These chemigenetic fluorescent indicators for calcium ions were used in rat hippocampal neuron cultures with a field electrode and show that they work with several different small-molecule dyes (FIG. 5A-5B, FIG. 6A-6D, FIG. 7A-7C).

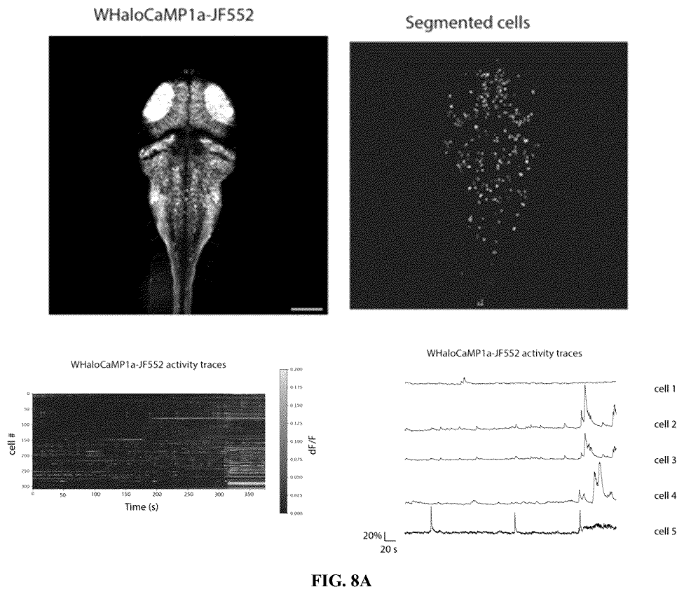

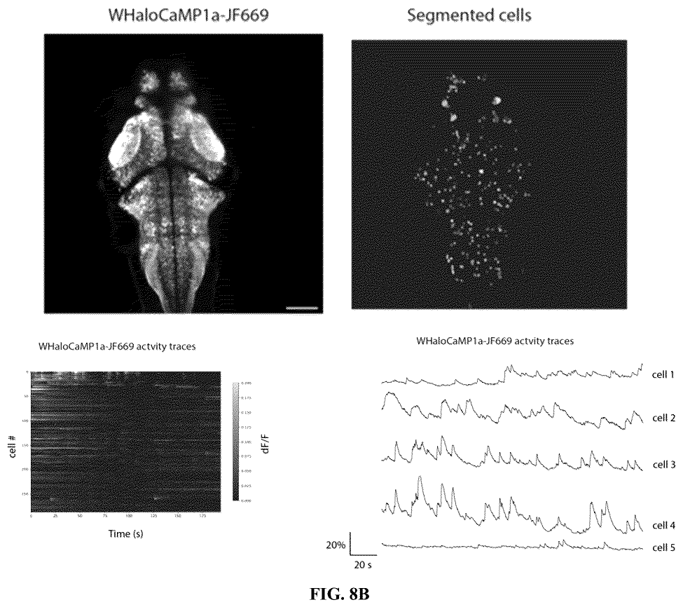

These chemigenetic fluorescent indicators for calcium ions were used to record neuronal activity in zebrafish larvae (FIG. 8A-8B).

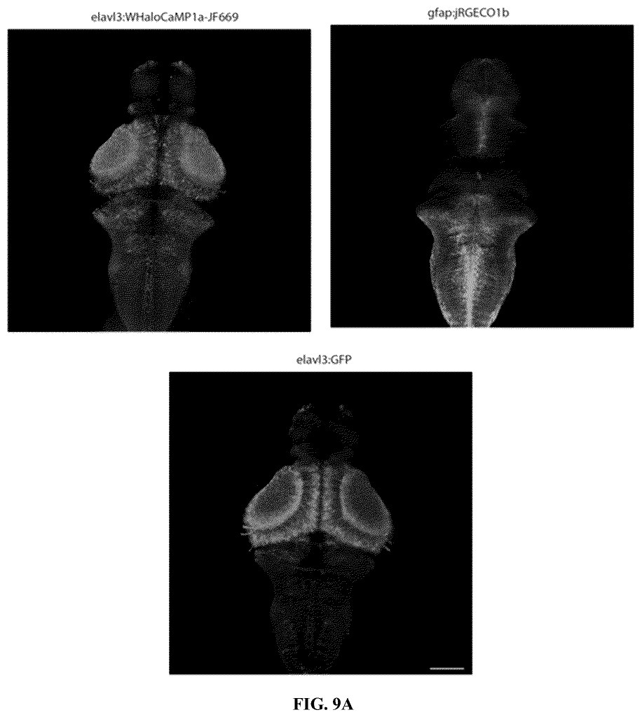

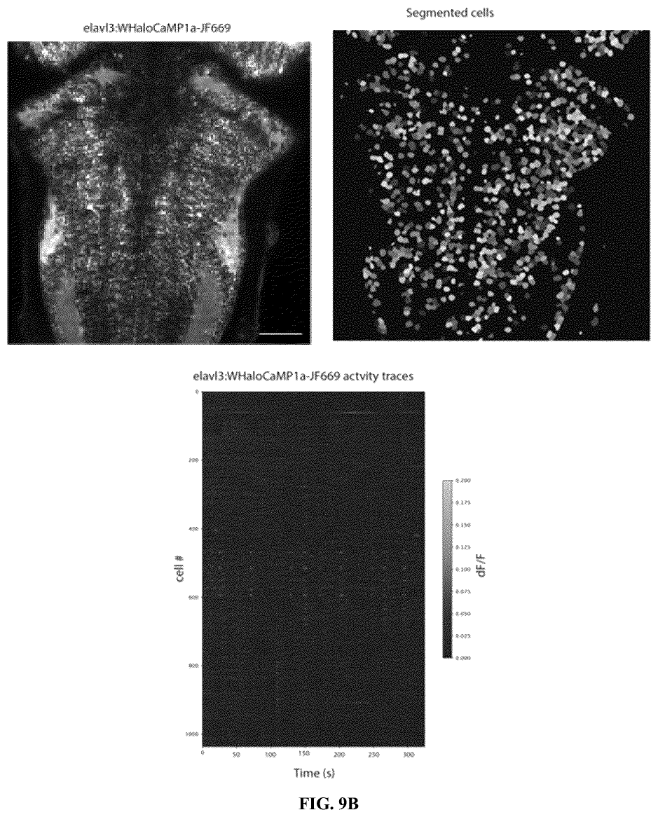

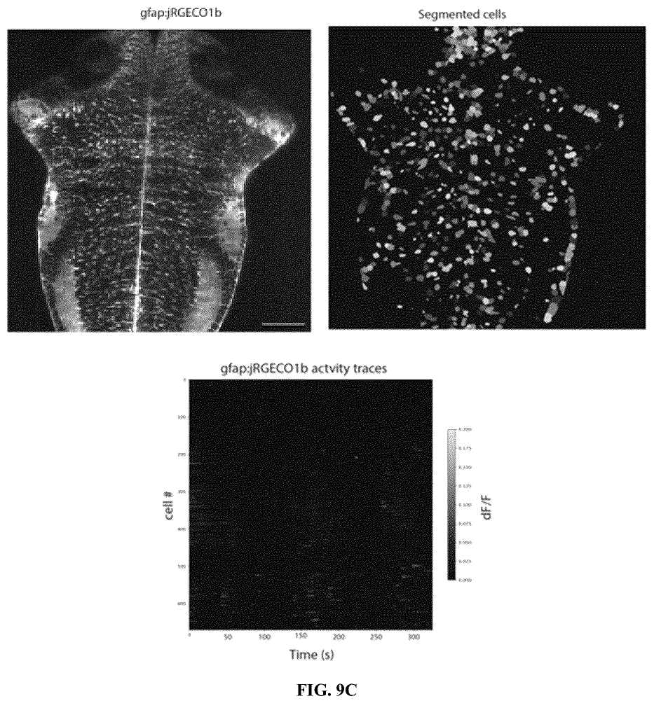

It was also shown that dual color functional imaging can be performed in live zebrafish larvae using a chemigenetic fluorescent indicator for calcium ions. In particular, it was shown that a chemigenetic fluorescent indicator including a far-red small-molecule fluorescent dye could be used in neurons together with the known red calcium indicator jRGECO1b in astrocytes (FIG. 9A-9C).

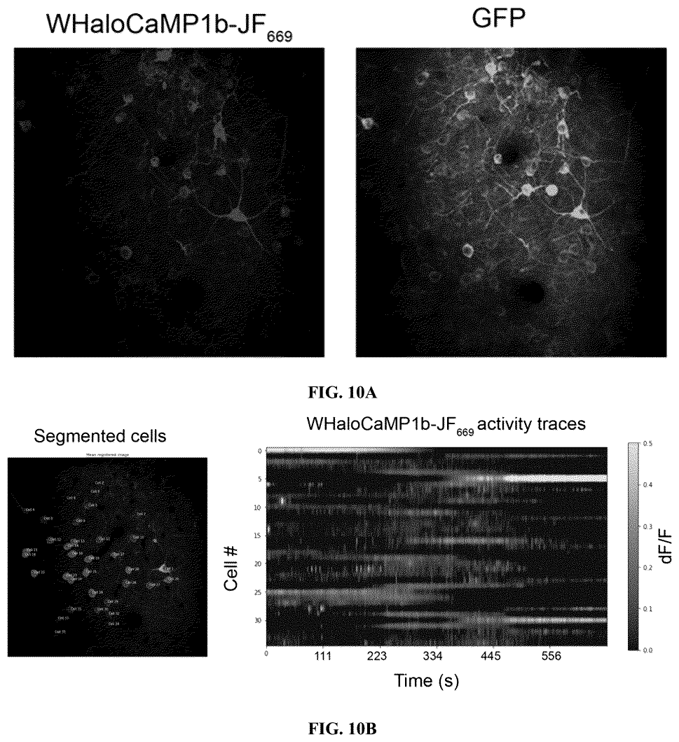

Additionally, it was shown that the chemigenetic fluorescent indicators for calcium ions can be assembled in mouse cortex after retro orbital injection of the small-molecule ligand, and record changes in fluorescent intensity (FIG. 10A-10B).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. A. Wang, J. Feng, Y. Li, P. Zou, Beyond fluorescent proteins: hybrid and bioluminescent indicators for imaging neural activities. ACS Chemical Neuroscience 9, 639-650 (2018).
2. T. Ueda, T. Tamura, I. Hamachi, In situ construction of protein-based semisynthetic biosensors. ACS Sensors 3, 527-539 (2018).
3. B. L. Sabatini, L. Tian, Imaging neurotransmitter and neuromodulator dynamics in vivo with genetically encoded indicators. Neuron 108, 17-32 (2020).
4. J. B. Grimm et al., A general method to improve fluorophores for live-cell and single-molecule microscopy. Nature Methods 12, 244-250 (2015).
5. C. Deo et al., The HaloTag as a general scaffold for far-red tunable chemigenetic indicators. Nature Chemical Biology 17, 718-723 (2021).
6. H. Farrants, A. G. Tebo, Fluorescent chemigenetic actuators and indicators for use in living animals. Current Opinion in Pharmacology 62, 159-167 (2022).
7. J. B. Grimm et al., A general method to optimize and functionalize red-shifted rhodamine dyes. Nature Methods 17, 815-821 (2020).
8. G. V. Los et al., HaloTag: A novel protein labeling technology for cell imaging and protein analysis. ACS Chemical Biology 3, 373-382 (2008).
9. M. S. Frei et al., Engineered HaloTag variants for fluorescence lifetime multiplexing. Nature Methods 19, 65-70 (2022).
10. M. Mita et al., Green Fluorescent Protein-Based Glucose Indicators Report Glucose Dynamics in Living Cells. Analytical Chemistry 91, 4821-4830 (2019).
11. Y. Zhang et al., Fast and sensitive GCaMP calcium indicators for imaging neural populations. bioRxiv, 2021.2011.2008.467793 (2021).
12. B. Moeyaert et al., Improved methods for marking active neuron populations. Nature Communications 9, 4440 (2018).
13. Nasu et al., Structure- and mechanism-guided design of single fluorescent protein-based biosensors. Nature Chem Bio 17, 509 (2021).
14. Akerbom et al. Genetically encoded calcium indicators for multi-color neural activity imaging and combination with optogenetics. Front. Mol. Neurosci 6, 1 (2013).
15. Shen et al. A genetically encoded Ca2+ indicator based on circularly permutated sea anemone red fluorescent protein eqFP578, BMC Biology (2018).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A chemigenetic fluorescent indicator for detecting an analyte, comprising:
(a) an analyte sensing domain (ASD),
(b) a self-labeling protein (SLP) having a tryptophan-modification, provided together with the ASD in a fusion protein, and
(c) a bioavailable fluorescent dye conjugated to a ligand for the SLP.

2. The indicator of claim 1, wherein the tryptophan-modification is positioned relative to the ASD and the fluorescent dye, upon binding of the ligand to the SLP, such that the tryptophan-modification migrates to quench or allow fluorescence when the ASD undergoes a conformational change when it binds to an analyte.

3. The indicator of claim 1, wherein the tryptophan-modification in the SLP is within about 5 Å from the fluorescent dye, upon binding of ligand to the SLP.

4. The indicator of claim 1, wherein the SLP has the sequence of SEQ ID NO: 1;
provided as an N-terminal portion and a C-terminal portion;
wherein 0, 1, 2, 3, 4, or 5 residues are removed from a terminus of the N-terminal portion, the C-terminal portion, or both; and
wherein there are 1, 2, 3, 4, or 5 mutations between residues 145 and 180.

5. The indicator of claim 4, wherein the tryptophan-modification is between residues 145 and 176.

6. The indicator of claim 5, wherein the tryptophan-modification is at residue 151 or 171.

7. The indicator of claim 5, wherein there is at least one additional mutation at a residue selected from the group consisting of: 157, 158, 176, 178, and 180.

8. The indicator of claim 7, wherein the at least one additional mutation is selected from the group consisting of: V157L, G158D, G176A, V178A, V178I, G178W, P180Y, P180T, and P180V.

9. The indicator of claim 4, wherein the ASD and/or a linker is disposed between the N-terminal and C-terminal portions of the SLP.

10. The indicator of claim 9, wherein
the N-terminal portion of the SLP extends from residue 1-4 to residue 150-180; and
the C-terminal portion of the SLP extends from residue 151-181 to 294-297.

11. The indicator of claim 4, wherein the SLP is circularly permutated.

12. The indicator of claim 11, wherein a linker is disposed between the C-terminal and the N-terminal portions of the SLP.

13. The indicator of claim 1, wherein the ASD undergoes a conformational change when it binds to an analyte.

14. The indicator of claim 13, wherein the analyte is selected from the group consisting of: calcium (Ca²⁺), glucose, GABA, DA, NE, opioids, 5-HT, MT, Ach, maltose, Tre, ATP, GTP, cAMP, cGMP, ADP, citrate, pyruvate, NAD+/NADH, NADPH, PKA, PKC, Pn, nicotine, H2O2, NH4+, and Zn2+.

15. The indicator of claim 13, wherein the analyte is Ca²⁺.

16. The indicator of claim 15, wherein the ASD comprises calmodulin and a calmodulin binding peptide.

17. The indicator of claim 16, wherein there is a linker disposed between the calmodulin and a calmodulin binding peptide.

18. The indicator of claim 13, wherein the analyte is glucose.

19. The indicator of claim 18, wherein the ASD comprises glucose binding protein (MgIB).

20. A method of detecting an analyte-of-interest in a cell, comprising: contacting the cell with the indicator of claim 1.


SEQUENCE LISTING (18 sequences)

SEQ ID NO: 1 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 2 - protein, 147 aa, synthetic construct
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 3 - protein, 20 aa, synthetic construct
ARRKWQKTGHAVRAIGRLSS

SEQ ID NO: 4 - protein, 19 aa, synthetic construct
RKKTFKEVANAVKISASLM

SEQ ID NO: 5 - protein, 307 aa, synthetic construct
VDTSIGVTIYKYDDNYMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFS

SEQ ID NO: 6 - protein, 476 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEWTLPMAVARARRKWQKTGHATRAIGRLSSGGSGGSGGSGGSDQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKYLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 7 - DNA, 1428 bp, synthetic construct
  source 1..1428: mol_type = other DNA
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagtggacgctgccgatggccgtcgcgcgcgcccgtcgtaaatggcagaaaacaggccatgcgacgcgtgctatcggtcgtttgtcgagcggaggctctggcggttccggcggttcaggtggatcagatcaattaacagaggaacagattgcggagtttaaggaagcgttctctttatttgataaggatggcgacggtacaatcactactaaagaattgggaacagtcatgcgctcattggggcaaaatccgacagaggctgaattgcaggacatgattaacgaggtagacgccgatgggaacgggactatcgactttccggaatttcttactatgatggcacgcaaaatgaaagataccgattctgaagaagagatccgtgaagccttccgtgtttttgataaagatgggaacggctacatcagtgctgctgagttacgccatgtgatgacaaatctgggggaaaaacttaccgacgaagaagtagacgaaatgattcgcgaggcggatattgacggggatggacaagtaaactacgaggaatttgtgcagatgatgaccgccaagtatctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggc

SEQ ID NO: 8 - protein, 481 aa, synthetic construct
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKVLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGTGGSGGTGGSGGTGGSAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEWTLPMGVWRARRKWQKTGHAVRAIGRLSS

SEQ ID NO: 9 - DNA, 1446 bp, synthetic construct
  source 1..1446: mol_type = other DNA
atggatcaattaacagaggaacagattgcggagtttaaggaagcgttctctttatttgataaggatggcgacggtacaatcactactaaagaattgggaacagtcatgcgctcattggggcaaaatccgacagaggctgaattgcaggacatgattaacgaggtagacgccgatgggaacgggactatcgactttccggaatttcttactatgatggcacgcaaaatgaaagataccgattctgaagaagagatccgtgaagccttccgtgtttttgataaagatgggaacggctacatcagtgctgctgagttacgccatgtgatgacaaatctgggggaaaaacttaccgacgaagaagtagacgaaatgattcgcgaggcggatattgacggggatggacaagtaaactacgaggaatttgtgcagatgatgaccgccaaggtgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggcggaggaacaggtggttctggtggaacagggggtagcggaggtacaggaggaagtgcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgctccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagtggacgctgccgatgggtgtctggcgcgcccgtcgtaaatggcagaaaacaggccatgcggttcgtgctatcggtcgtttgtcgagc

SEQ ID NO: 10 - protein, 475 aa, synthetic construct
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKLDRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSGGTGGSGGTGGSAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQWFRTARRKWQKTGHAVRAIGRLSS

SEQ ID NO: 11 - DNA, 1428 bp, synthetic construct
  source 1..1428: mol_type = other DNA
atggatcaattaacagaggaacagattgcggagtttaaggaagcgttctctttatttgataaggatggcgacggtacaatcactactaaagaattgggaacagtcatgcgctcattggggcaaaatccgacagaggctgaattgcaggacatgattaacgaggtagacgccgatgggaacgggactatcgactttccggaatttcttactatgatggcacgcaaaatgaaagataccgattctgaagaagagatccgtgaagccttccgtgtttttgataaagatgggaacggctacatcagtgctgctgagttacgccatgtgatgacaaatctgggggaaaaacttaccgacgaagaagtagacgaaatgattcgcgaggcggatattgacggggatggacaagtaaactacgaggaatttgtgcagatgatgaccgccaagctggatcgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggcggcagcgggggcacaggcgggagtggaggcaccggcggatcagcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccagtggttccgcaccgcccgtcgtaaatggcagaaaacaggccatgcggttcgtgctatcggtcgtttgtcgagc

SEQ ID NO: 12 - protein, 608 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQWFRTGGSVDTSIGVTIYKYDDNYMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSGGSVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 13 - DNA, 1824 bp, synthetic construct
  source 1..1824: mol_type = other DNA
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccagtggttccgcaccggcggttcagttgatactagcattggtgtaacaatctataagtacgacgataactatatgtctgtagtgcgcaaggctattgagcaagatgcgaaagccgcgccagatgttcagctgctgatgaatgattctcagaatgaccagtccaagcagaacgatcagatcgacgtattgctggcgaaaggggtgaaggcactggcaatcaacctggttgacccggcagctgcgggtacggtgattgagaaagcgcgtgggcaaaacgtgccggtggttttcttcaacaaagaaccgtctcgtaaggcgctggatagctacgacaaagcctactacgttggcactgactccaaagagtccggcattattcaaggcgatttgattgctaaacactgggcggcgaatcagggttgggatctgaacaaagacggtcagattcagttcgtactgctgaaaggtgaaccgggccatccggatgcagaagcacgtaccacttacgtgattaaagaattgaacgataaaggcatcaaaactgaacagttacagttagataccgcaatgtgggacaccgctcaggcgaaagataagatggacgcctggctgtctggcccgaacgccaacaaaatcgaagtggttatcgccaacaacgatgcgatggcaatgggcgcggttgaagcgctgaaagcacacaacaagtccagcattccggtgtttggcgtcgatgcgctgccagaagcgctggcgctggtgaaatccggtgcactggcgggcaccgtactgaacgatgctaacaaccaggcgaaagcgacctttgatctggcgaaaaacctggccgatggtaaaggtgcggctgatggcaccaactggaaaatcgacaacaaagtggtccgcgtaccttatgttggcgtagataaagacaacctggctgagttcagcggtggctctgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggc

SEQ ID NO: 14 - protein, 609 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEWTLPMGVVRLLGVDTSIGVTIYKYDDNYMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSEVPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 15 - DNA, 1827 bp, synthetic construct
  source 1..1827: mol_type = other DNA
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagtggacgctgccgatgggtgtcgtccgcctcctgggagttgatactagcattggtgtaacaatctataagtacgacgataactatatgtctgtagtgcgcaaggctattgagcaagatgcgaaagccgcgccagatgttcagctgctgatgaatgattctcagaatgaccagtccaagcagaacgatcagatcgacgtattgctggcgaaaggggtgaaggcactggcaatcaacctggttgacccggcagctgcgggtacggtgattgagaaagcgcgtgggcaaaacgtgccggtggttttcttcaacaaagaaccgtctcgtaaggcgctggatagctacgacaaagcctactacgttggcactgactccaaagagtccggcattattcaaggcgatttgattgctaaacactgggcggcgaatcagggttgggatctgaacaaagacggtcagattcagttcgtactgctgaaaggtgaaccgggccatccggatgcagaagcacgtaccacttacgtgattaaagaattgaacgataaaggcatcaaaactgaacagttacagttagataccgcaatgtgggacaccgctcaggcgaaagataagatggacgcctggctgtctggcccgaacgccaacaaaatcgaagtggttatcgccaacaacgatgcgatggcaatgggcgcggttgaagcgctgaaagcacacaacaagtccagcattccggtgtttggcgtcgatgcgctgccagaagcgctggcgctggtgaaatccggtgcactggcgggcaccgtactgaacgatgctaacaaccaggcgaaagcgacctttgatctggcgaaaaacctggccgatggtaaaggtgcggctgatggcaccaactggaaaatcgacaacaaagtggtccgcgtaccttatgttggcgtagataaagacaacctggctgagttcagcgaggtgccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggc

SEQ ID NO: 16 - protein, 12 aa, synthetic construct
GGSGGSGGSGGS

SEQ ID NO: 17 - protein, 15 aa, synthetic construct
GGSGGTGGSGGTGGS

SEQ ID NO: 18 - protein, 19 aa, synthetic construct
GGGTGGSGGTGGSGGTGGS